(19) United States
(12) Patent Application Publication
(10) Pub. No.: US 2005/0203318 A1
(43) Pub. Date: Sep. 15, 2005

(54) Catalyst and method for the preparation of chloroform and chlorinated paraffins

(75) Inventors: Vladimir V. Smirnov, Moscow (RU); Tarkhanova Irina Gennadyevna, Moscow (RU); Nevskaya Svetlana Mikhailovna, Moscow (RU); Zelikman Vladimir Mendeleevich, Moscow (RU)

Correspondence Address: Reed Smith LLP;East Tower-Suite 1100, 1301 K Street, N.W., Washington, DC, 20005-3373, US

(73) Assignee: NeurOK LLC

(21) Appl. No.: 10/797,610

(22) Filed: Mar. 11, 2004

(51) Int. Cl. C07C 17/361

(52) U.S. Cl. 570/260

(57) ABSTRACT

This invention relates to a catalyst and process for simultaneous preparation of chloroform and chlorinated paraffins from carbon tetrachloride and paraffins. The catalyst is a liquid preparation of a complex of copper compound and nitrogen-containing organic compound in a liquid phase base. In embodiments of the invention, the catalyst includes 1) a copper(I) or copper(II) compound; 2) a tertiary ammonium salt, amino acid, amide, alkanolamine, urea, or derivative thereof; and 3) an alcohol, hydroxyl-containing organic compound, or water. The process for the preparation of chloroform and chlorinated paraffins according to the present invention includes hydrogenating carbon tetrachloride by one or more n-paraffins in a liquid phase at 150-170° C. in the presence of the catalyst. The hydrogenating is carried out at a molar ratio of carbon tetrachloride to paraffin equal to at least approximately 1:1 with the catalyst present in an amount equal to approximately 1-10% by volume.

### FIELD OF THE INVENTION

The invention relates to processes for simultaneous preparation of chloroform and chlorinated paraffins, and to catalysts used in the processes.

### BACKGROUND OF THE INVENTION

Chloroform, also known as trichloromethane, represented by the chemical formula CHCl₃, is a well known chemical compound that is useful as an intermediate in the production of various useful alkyl halides. Such alkyl halides include refrigerants (such as ozone-safe alternatives to freon), solvents, and pesticides. Larger chain halogenated alkanes such as chlorinated high carbon alkanes are used as plastifiers for polymer materials. Polyvinylchloride, for example, is used in the preparation of plastic products, such as artificial leather and rubber-products, and as components of lacquers, lubricants, adhesives and coatings.

Carbon tetrachloride, also known as tetrachloromethane represented by the chemical formula CCl₄, is a well known chemical that has good solvent properties for cleaning and degreasing. It was once commonly used for dry cleaning, spot removing, and domestic cleaning chores, but has since been known to be highly toxic and carcinogenic and is no longer used for such purposes. Carbon tetrachloride is a main byproduct in the principal reactions used for the synthesis of chloromethanes from methane and chlorine, and it is considered in such cases, largely as a waste product that must be disposed of in a safe and ecologically friendly manner. However, its disposal by methods such as incineration is hazardous and risky considering the environmental and health issues and the wasting of chlorine.

An alternative method for the disposition of carbon tetrachloride involves the catalytic dechlorination and hydrogenation of carbon tetrachloride into chloroform or other useful chlorinated methanes, such as dichloromethane or chloromethane.

Various reactions for catalytic dechlorination and hydrogenation have been identified. One known reaction for hydrogenating carbon tetrachloride reacts the carbon tetrachloride with C₁₀ to C₁₅ alkanes (alkanes also being referred to herein as “paraffins”) to produce both chloroform and chlorinated alkanes. This reaction is described in Russian patent 2,107,544 (published Mar. 27, 1998). The reaction involves reacting carbon tetrachloride with one or more paraffins in a liquid phase in the presence of a copper catalyst at approximately 150-200° C. for between 3-16 hours. The disclosed catalyst is a product of reacting cuprous (copper(I)) chloride with caprolactam or dimethylformamide formed on a carrier vehicle of silica gel. The content of cuprous chloride present is approximately 1-7% by weight in comparison to the weight of the vehicle. In the catalyst, the molar ratio of cuprous chloride to dimethylformamide or caprolactam is 1:50-200.

A disadvantage of this catalytic reaction is the poor quality of the chlorinated paraffin prepared in the reaction. The chlorinated paraffins of this reaction comprise resins having intense coloration. Such paraffins are less commercially desirable. Vacuum distillation of the resulting reaction mass is necessary to prepare a commercial product, making the process more complicated and more expensive. Further, the selectivity of the reaction for chloroform as a reaction product does not exceed 97%, with conversion of paraffins in the range of 70-80%. Improved selectivity would be desirable at high conversion rates of the paraffins.

A similar process for preparing chloroform from carbon tetrachloride is disclosed in Russian patent 2,107,678 (published Mar. 27, 1998). This process hydrogenates carbon tetrachloride with a C₁₀-C₁₅ paraffin or mixture of such paraffins in the presence of a catalyst, which is a complex compound of copper(I) with caprolactam or dimethlylformamide on a solid vehicle. The reaction is conducted at 150-200° C. with time of a contact of approximately 3-16 hours at a molar ratio of carbon tetrachloride and paraffin in the range of (2-6):1.

The process also suffers from a poor quality of the prepared chlorinated paraffin. The chlorinated paraffin product contains resins and is intensely colored. Also, expensive vacuum distillation of the reaction mass is necessary to obtain a commercial product. Further, the selectivity of the process for chloroform is also unsatisfactory, reaching only 95-97% at high conversion of carbon tetrachloride. Higher selectivity values can be obtained only at significantly lower degrees of carbon tetrachloride conversion.

Another known process for preparing chloroform and chlorinated paraffins by hydrogenating carbon tetrachloride using n-paraffins C₁₀-C₁₅ or mixtures thereof, in a liquid phase, is disclosed in Russian patent 2,187,489 (published Aug. 8, 2002). This reaction takes place at 150-180° C. in the presence of a copper-containing catalyst. The catalyst is a complex of copper chloride with quaternary ammonium salt chlorides of the general formulas:
(R)₃(R¹)NCl, or
(R)₂(R¹)₂NCl,
where R is C₁-C₁₈ alkyl, R¹ is C₁-C₁₈ alkyl, benzyl or salt of the formula:
(R)—CH[N(R¹)₃CH[N(R¹)₃-(R²)Cl₂],
where R² is hydrogen or a low alkyl and where R and R¹ are as indicated above. A soluble catalyst is formed with the molar ratio of ammonium tertiary salts and copper in the range of (1-10):1.

The main disadvantage of this process is the low quality of main products, which have a high content of tarry substances. Again, high-vacuum purification is necessary, making the process complicated and expensive, and, therefore, commercially undesirable in many circumstances.

European Patent No. 1,201,300 (published May 2, 2001) discloses a process for preparing chloroform from carbon tetrachloride in a fluidized-bed hydrochlorination reaction in the presence of a platinum catalyst. This process is not ideal in that it requires the production of a complex catalyst for use in a fluidized bed. Usage of a precious metal like platinum in a catalyst increases the cost of the process. Further, the reaction uses gaseous hydrogen at temperatures in the range of 90-170° C., risking explosion and fire for the combustible hydrogen gas. Importantly, this reaction also provides a relatively low selectivity for chloroform, approximately 85% and below, and has a high incidence of chlorinated hydrogen and methane byproducts that are contaminated by organic substances that have no commercial value.

U.S. Pat. No. 5,334,782 describes a process for dechlorinating chlorinated methanes, and carbon tetrachloride in particular, in a liquid phase reaction. The reaction takes place in the presence of a catalyst consisting of an elemental metal selected from the group consisting of ruthenium, rhodium, palladium and platinum, and, optionally, a second elemental metal being selected from the group consisting of copper, silver, and gold. The process is a liquid phase fixed bed reaction carried out at 0° to 200° C. with hydrogen. This process has the disadvantages of using precious metals in a complex catalyst, and does not use the chlorine freed from the chlorinated methanes in a productive manner.

In view of the current techniques for converting carbon tetrachloride into chloroform and for chlorinating n-paraffins, it would be desirable to have an improved process that does not suffer the disadvantages identified above.

### SUMMARY OF THE INVENTION

Accordingly, the present invention is directed to a process that substantially obviates one or more of the problems due to limitations and disadvantages of the related art. Additional features and advantages of the invention will be set forth in the description which follows, and in part will be apparent from the description, or may be learned by practice of the invention. The objectives and other advantages of the invention will be realized and attained by the means particularly pointed out in the written description and claims hereof as well as in the appended drawings.

The present invention provides a catalytic process for simultaneously producing chloroform from carbon tetrachloride and producing chlorinated paraffins of high quality. The process as described herein is simple, and provides high yield of target products.

Embodiments of the present invention utilize a liquid phase catalyst reaction for the preparation of chloroform and chlorinated paraffins from carbon tetrachloride and paraffins. The catalyst is a complex of a copper compound with a nitrogen-containing organic compound in a liquid phase base. The copper compound is a copper(I) salt, such as chloride, bromide or carboxylate, or a copper(II) salt, such as chloride, bromide or carboxylate. The nitrogen-containing organic compound is a quaternary ammonium salt, amino acid, amide, alkanolamine, urea, or derivative thereof. The liquid phase base for the catalyst reaction can be an alcohol, a hydroxyl-containing organic compound, or water.

In embodiments of the invention, the catalyst is prepared according to the following proportions of the components (wt %):

| | | | |
|---|---|---|---|
| i: | copper(I) or copper(II) salt: | 1.5-4.0 | wt % |
| ii: | nitrogen-containing organic | 30.0-50.0 | wt % |
| | compound: | | |

| | | |
|---|---|---|
| iii: | liquid phase base: | balance |

The process for preparing chloroform and chlorinated paraffins according to embodiments of the invention comprises hydrogenating carbon tetrachloride by an n-paraffin, or mixture of different n-paraffins, in a liquid phase at least at approximately 150° C., and preferably between 150-170° C., in the presence the catalyst, followed by subsequent distillation of chloroform and recovery of chlorinated n-paraffins. The catalyzed hydrogenating reaction is carried out at a molar ratio of carbon tetrachloride and paraffin equal to at least 1:1, and preferably in the range of 1:1 to 2:1, and most preferably approximately 1.5:1, the catalyst is present in an amount equal to approximately 1-10% by volume. The hydrogenation reaction can be carried out for approximately 3-12 hours, and preferably for approximately 6-8 hours.

The above-described invention provides an improvement to the quality of chlorinated paraffin prepared over prior art methods, and leads to high yield and selectivity for chloroform. The chlorinated paraffin products are colorless or have mild yellowish or brownish coloring, are substantially free of tarry matters and hydrogen chloride, and are of suitable quality for direct usage in further processing techniques without additional purification. Complex steps for purification and refinement necessary in prior art methods, including vacuum distillation and flashing, are eliminated, thereby reducing the cost of the produced chlorinated paraffin and simplifying downstream processing.

Furthermore, selectivity of conversion of carbon tetrachloride to chloroform has been found to be at or near 100%. Selectivity of chloroform approaching 100% can greatly simplify any subsequent steps necessary to separate chlorinated methane products from reactants.

It is to be understood that both the foregoing general description and the following detailed description are exemplary and explanatory and are intended to provide further explanation of the invention as claimed. Other objects and features of the invention will become apparent from the following detailed description considered in conjunction with the accompanying drawings. The accompanying drawings are included solely for purposes of illustration and not as a definition of the limits of the invention.

### BRIEF DESCRIPTION OF THE DRAWINGS

FIG. 1 is a chart depicting results from various experiments with different catalysts according to the present invention.

FIG. 2 is a chart depicting results from various experiments with different catalysts according to the present invention in addition to the experimental results depicted in FIG. 1.

### DETAILED DESCRIPTION OF THE PREFERRED EMBODIMENTS

Embodiments of the present invention utilize a liquid phase catalyst reaction for the preparation of chloroform and chlorinated paraffins products from carbon tetrachloride and paraffins. The catalyst is a complex of a copper compound with a nitrogen-containing organic compound in a liquid phase base. The copper compound is a copper(I) salt, such as chloride, bromide, carboxylate or other ion of similar properties, or a copper(II) salt, such as chloride, bromide, carboxylate or other ion of similar properties. The nitrogen-containing organic compound is a tertiary ammonium salt, amino acid, amide, alkanolamine, urea, or a derivative thereof. The liquid phase base for the catalyst reaction can be an alcohol, a hydroxyl-containing organic compound, or water.

In embodiments of the invention, the catalyst is prepared according to the following proportions of the components (wt %):

| | | | |
|---|---|---|---|
| i: | copper(I) or copper(II) salt: | 1.5-4.0 | wt % |
| ii: | nitrogen-containing organic | 30.0-50.0 | wt % |
| | compound: | | |

| | | |
|---|---|---|
| iii: | liquid phase base: | balance |

The process for preparing chloroform and chlorinated paraffins according to embodiments of the invention comprises hydrogenating carbon tetrachloride by an n-paraffin, or mixture of different n-paraffins, in a liquid phase at least at 150° C., and preferably at approximately 150-170° C., in the presence the catalyst, followed by subsequent distillation of chloroform and recovery of chlorinated n-paraffins. The catalyzed hydrogenating reaction is carried out at a molar ratio of carbon tetrachloride to paraffin equal to at least 1:1, and preferably between 1:1 and 2:1. The process is conducted in the presence of 1-10% catalyst by volume. The hydrogenation reaction can be carried out for 3-12 hours, and preferably is run for approximately 6-8 hours.

The catalyst in one embodiment of the present invention contains a nitrogen-containing organic compound in the form of a quaternary ammonium salt of the formula [NR¹R²R³R⁴]⁺X⁻, where R¹, R², R³ and R⁴ can be different or identical functional groups selected from alkyls, arylalkyls, cycloalkyls, and hydroxyalkyls. Accordingly, ion X⁻ in the ammonium tertiary salt can be a chloride, bromide, alcoholate, or other ion of similar properties.

In another embodiment of the invention, the catalyst contains nitrogen-containing organic compounds in the form of amino acids selected from threonine, asparagine, hydroxyproline, betaine, cysteine, and serine.

In yet another embodiment, the catalyst contains a nitrogen-containing organic compound in the form of an amide selected from formamide, acetamine, dimethylformamide, dimethylacetamide, urea and caprolactam.

In other embodiments, the catalyst contains nitrogen-containing organic compounds in the form of an alkanamine selected from ethanolamine, di-ethanolanime, tri-ethanolamine, and hydroxides thereof, and di-(2-hydroxyethyl) dodecylamine.

Optionally, the catalyst can contain a mixture of at least two nitrogen-containing organic compounds of the above listed groups.

In all embodiments of the invention, the copper compound and nitrogen-containing organic compound are prepared in a liquid phase reaction environment. The liquid phase base, in embodiments of the invention wherein it is an alcohol, can be selected from the group consisting of methanol, ethanol, and isopropanol. Additionally, the liquid reaction environment can be an aromatic alcohol such as phenol or alkylphenol.

The liquid reaction environment can also comprise water or mixtures of water and/or one or more alcohols.

The process for preparing chloroform and chlorinated paraffins according to embodiments of the invention comprises hydrogenating carbon tetrachloride by an n-paraffin, or mixture of different n-paraffins, in a liquid phase at least at approximately 150° C., and preferably at 150-170° C., in the presence the catalyst, followed by subsequent distillation of chloroform and recovery of chlorinated n-paraffins. The catalyzed hydrogenating reaction is carried out at a molar ratio of carbon tetrachloride and paraffin equal at least 1:1, and preferably between approximately 1:1 and 2:1, and is conducted in the presence of 1-10% catalyst by volume. The hydrogenation reaction can be carried out for 3-12 hours, and typically for between about 6-8 hours.

The invention will now be illustrated with respect to several examples.

### EXAMPLE 1

Preparation of the catalyst starts with measuring 1 g of CuCl₂ and adding that to a vessel containing a mixture of 10 ml of water, 20 ml of ethyl alcohol (97% ethanol in this experiment). Next, 12 g of the nitrogen-containing organic compound, a quaternary ammonium salt of the formula [NR¹R²R³R⁴]⁺X⁻, where R¹ and R² are methyl groups, R₃ is a benzyl group, R₄ is a dodecyl group, and X⁻ is a Cl⁻ ion) is measured out and added to the liquid. The salt is dissolved, leading to the liquid turning an intense green color. As a result, a liquid phase catalyst environment (“Catalyst A”) of the following composition is obtained:

| | | |
|---|---|---|
| CuCl₂ | 2.6 | wt % |
| Dimethylbenzyldodecylammoniumchloride | 30.9 | wt % |
| Water | 25.8 | wt % |
| Ethanol | 40.7 | wt % |

Using steps similar to as described above in Example 1, the catalyst environments of the following compositions were likewise obtained:

| | | | |
|---|---|---|---|
| B. | CuCl | 3 | wt % |
| | Tributylbenzylchloride | 47 | wt % |
| | Ethanol | 50 | wt % |
| C. | CuCl₂ | 2.5 | wt % |
| | Tetrabutanolchloride | 30 | wt % |
| | Propanol | 67.5 | wt % |
| D. | CuCl₂ | 3 | wt % |
| | Tributylbenzylchloride | 47 | wt % |
| | Water | 50 | wt % |
| E. | CuCl₂ | 2.5 | wt % |
| | Cysteine | 47.5 | wt % |
| | Ethanol | 50 | wt % |
| F. | CuCl₂ | 3.5 | wt % |
| | Dimethylacetatamide | 46.5 | wt % |
| | Propanol | 50 | wt % |
| G. | CuCl₂ | 2.5 | wt % |
| | Caprolactam | 47.5 | wt % |
| | Isopropanol | 50 | wt % |
| H. | CuCl | 3 | wt % |
| | Urea | 42 | wt % |
| | Phenol | 55 | wt % |
| I. | Copper(I) acetate | 4 | wt % |
| | Tributylbensylammoniumchloride | 30 | wt % |
| | Diethanol amide | 20 | wt % |
| | water | 46 | wt % |
| J. | CuBr | 1.5 | wt % |
| | Tributylbenzylammoniumchloride | 5 | wt % |
| | Ethanol | 48.5 | wt % |
| K. | CuBr | 3 | wt % |
| | Tetrabutylammonium-ethoxide | 50 | wt % |
| | Ethanol | 47 | wt % |
| L. | CuCl₂ | 2.5 | wt % |
| | Di-(2-hydroxyethyl)dodecylamine | 50 | wt % |
| | Ethanol | 47.5 | wt % |
| M. | CuCl₂ | 2.5 | wt % |
| | Di-(2-hydroxyethyl)dodecylamine | 97.5 | wt % |

### EXAMPLE 2

In this example, 8 ml of carbon tetrachloride is reacted with 4 ml of a mixture of n-paraffins (2:1 by volume of carbon tetrachloride to paraffins). The mixture of n-paraffins (in weight %) is as described below in Table 1.

TABLE 1

| | |
|---|---|
| CH₃(CH₂)₁₁CH₃ (tridecane) | 13.3% |
| CH₃(CH₂)₁₂CH₃ (tetradecane) | 38.8% |
| CH₃(CH₂)₁₃CH₃ (pentadecane) | 33.7% |
| CH₃(CH₂)₁₄CH₃ (hexadecane) | 12.2% |
| CH₃(CH₂)₁₅CH₃ (heptadecane) | 2.0% |

The carbon tetrachloride and n-paraffin mixture is mixed in a vessel with 0.36 ml (2.9% vol.) of liquid Catalyst A (described above), and the vessel is sealed and placed into a glass autoclave. The autoclave is closed, purged by nitrogen, heated up to 160° C. and kept at this temperature for 6 hours.

Then the autoclave is cooled to room temperature and opened. Approximately 1.1 ml (1.6 g) of chloroform and 6.7 ml (10.7 g) of remaining unreacted carbon tetrachloride is recovered by distillation. The distillation residue of the reaction product is passed through a 10 cm layer of silica gel beads. A product consisting of 2.6 ml of chlorinated paraffin, in the form of a viscous liquid with light brownish tint, is obtained. Upon analysis, the chlorine content in the product makes 13.1 wt %. This chlorinated paraffin is of suitable quality for further commercial usage.

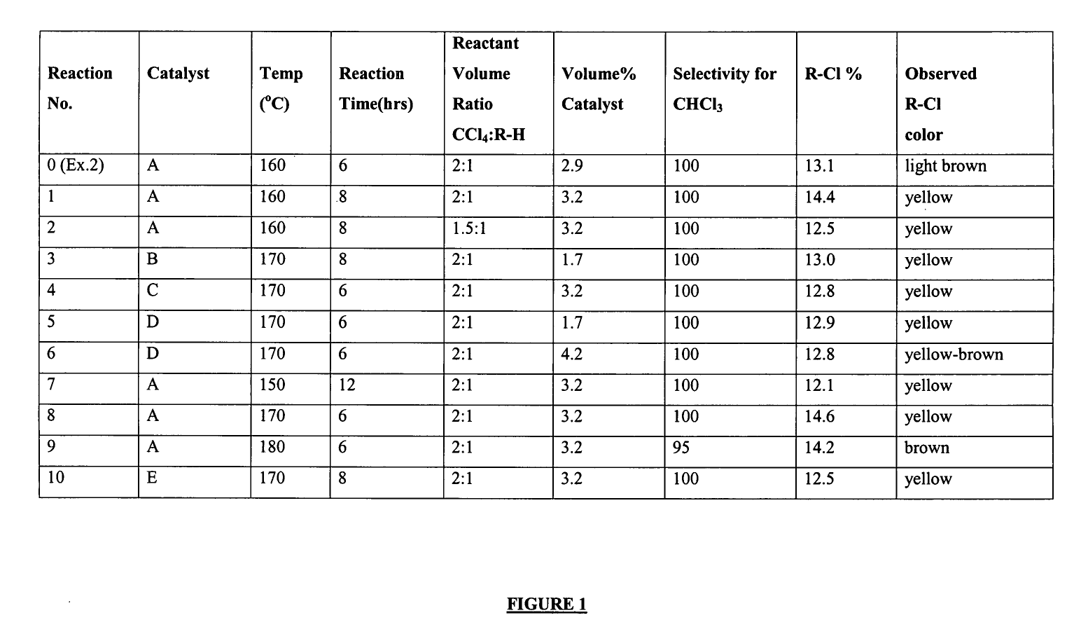

Analysis of the distillate indicates that selectivity of conversion of carbon tetrachloride to chloroform is 100%. The distillate is analyzed by gas liquid chromatography confirming that absence of any dichloromethane or chloromethane. The parameters and results for the experiment of Example 2 are summarized in the first row of the table depicted in FIG. 1.

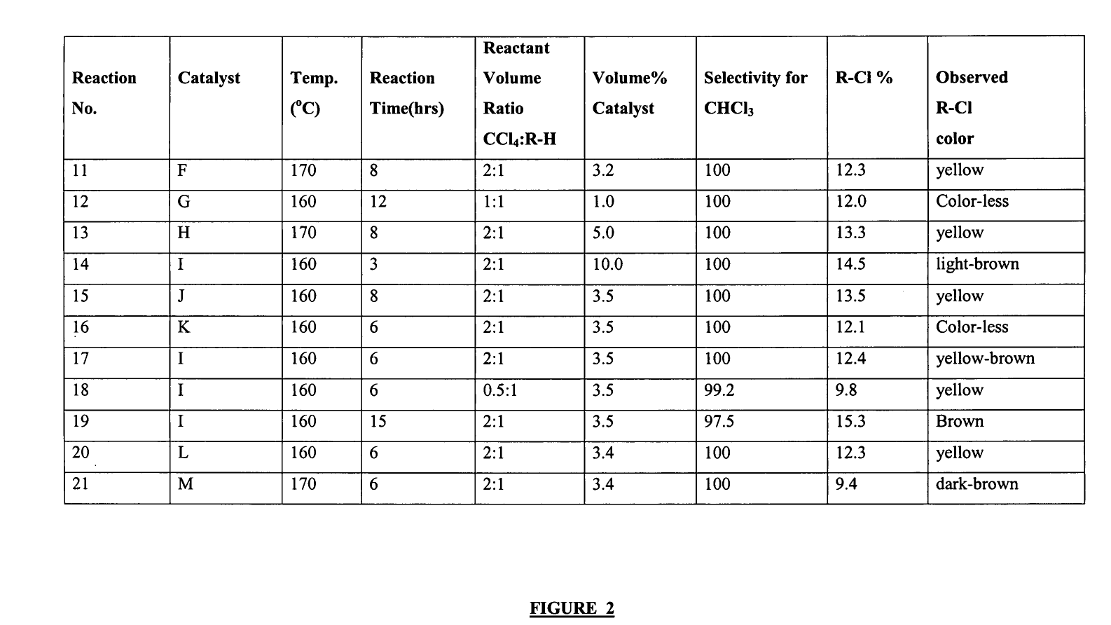

For the sake of comparison, similar experiments were carried out using the same n-paraffin mixture of Table 1 and according to the same basic methodology described in Example 2 above. These additional experiments utilized catalyst compositions A-M as described above and employed various relative concentrations of catalyst and reactants, various reaction temperatures, and various reaction times. The parameters and results of these additional experiments are shown in the table depicted as FIG. 1 and continued in the table depicted as FIG. 2.

As can be seen from the data depicted in the tables of FIG. 1 and FIG. 2, the process and catalysts according to the present invention are shown to be effective at least where the conditions are maintained between approximately 150-170° C. for approximately 3-12 hours. A presence of up to approximately 10% by volume of a catalyst according to the present invention at a molar ratio of carbon tetrachloride and paraffin equal to between approximately 0.5:1 and 2:1 leads to relatively high selectivity for the production of chloroform, relative to chloromethane and/or dichloromethane, and relatively high yield production of chlorinated paraffin products having acceptable quality for commercial use can be obtained with reactant ratios of at least approximately 1:1.

The experiments demonstrate that decreasing the relative amount of carbon tetrachloride reactant relative to paraffin reactant still results in a relatively high selectivity for chloroform, although this decrease tends to result in a decrease of the yield of chlorinated paraffin. (See, for example, Reaction Nos. 1 and 2 in FIG. 1, and Reaction Nos. 17 and 18 in FIG. 2). Further, increasing the temperature to above 170° C. for extended reaction times may be counterproductive for many catalysts as it may not increase yield significantly without an attendant considerable degradation of the color and quality of the chlorinated paraffin product (See Reaction Nos. 8 and 9). The experiments also demonstrate that the increase of the relative amount of the selected catalyst above approximately 10% by volume may not increase yield or selectivity. Further, reaction times exceeding approximately 12 hours may cause deterioration to the quality of the prepared chlorinated paraffin and/or decrease selectivity for chloroform. As may be deduced from the tables of FIG. 1 and FIG. 2, reaction times of 8 hours and less can provide acceptable selectivity for chloroform and yield of acceptable quality chlorinated paraffin. Therefore, reaction times of 6-8 hours can be preferred in circumstances for commercial viability reasons related to economies of scale. Similarly, it can be seen from the data in the tables that a volumetric proportion of carbon tetrachloride reactant relative to paraffin reactant greater than 1:1 and up to 2:1 can lead to increases in yield of chlorinated paraffin. Thus, reactant volumetric proportions of (1-2):1 of carbon tetrachloride to paraffin can be preferred in commercial circumstances where increased yield of chlorinated paraffins is desired.

Reaction No. 21, summarized in the table of FIG. 2, utilizes catalyst composition M, which, notably, does not contain a suitable liquid phase base. Catalyst composition M, in comparison to Catalyst composition L, contains additional di-(2-hydroxyethyl)dodecylamine (nitrogen-containing organic compound) in place of ethanol (liquid phase base). Comparison of Reactions Nos. 20 and 21 suggest that catalyst compositions falling outside of the compositions according to the present invention will produce inferior reaction products, in terms of both yield and quality of chlorinated paraffins.

Various preferred embodiments of the invention having been described above, one of ordinary skill in the art will readily appreciate that various insubstantial variations can be made to the preferred embodiments without departing from the spirit and scope of the present invention, which is defined by the appended claims and their equivalents.

1. A catalyst for the preparation of chloroform and chlorinated paraffins from carbon tetrachloride and paraffins, said catalyst comprising a complex of copper compound and nitrogen-containing organic compound present in a liquid phase base, wherein:
said copper compound is selected from the group consisting of a copper(I) salt and a copper(II) salt;
said nitrogen-containing organic compound is selected from the group consisting of a tertiary ammonium salt, an amino acid, an amide, an alkanolamine, urea, and derivatives thereof;
said liquid phase base is selected from the group consisting of an alcohol, a hydroxyl-containing organic compound, and water; and
wherein said catalyst comprises approximately 1.5-4.0% by weight copper compound and approximately 30.0-50.0% by weight nitrogen-containing organic compound with a remainder comprising liquid phase base.

2. The catalyst according to claim 1, wherein said nitrogen-containing organic compound is a quaternary ammonium salt of the formula [NR¹R²R³R⁴]⁺X⁻, wherein said R¹, R², R³ and R⁴ are functional groups selected from the group consisting of alkyls, arylalkyls, cycloalkyls, and hydroxyalkyls.

3. The catalyst according to claim 2, wherein said X⁻ is an ion selected from the group consisting of chloride, bromide, and alcoholate.

4. The catalyst according to claim 1, wherein said nitrogen-containing organic compound is an amino acid selected from the group consisting of threonine, asparagine, hydroxyproline, betaine, cysteine, and serine.

5. The catalyst according to claim 1, wherein said nitrogen-containing organic compound is an amide selected from the group consisting of formamide, acetatamide, dimethyl formamide, dimethylacetatcmide, caprolactam.

6. The catalyst according to claim 1, wherein said nitrogen-containing organic compound is urea.

7. The catalyst according to claim 1, wherein said nitrogen-containing organic compound is an alkanolamine selected from the group consisting of ethanolamine, di-ethanolamine, triethanolamine, hydroxides of ethanolamines, and di-(2-hydroxyethyl)dodecylamine.

8. The catalyst according to claim 1, wherein said catalyst comprises 30.0-50.0% by weight of a mixture of at least two nitrogen-containing organic compounds.

9. The catalyst according to claim 1, wherein said liquid phase base is an alcohol selected from the group consisting of methanol, ethanol, and isopropanol.

10. The catalyst according to claim 1, wherein said liquid phase base is an aromatic alcohol selected from the group consisting of phenol and alkylphenol.

11. The catalyst according to claim 1, wherein said copper containing compound is selected from the group consisting of a chloride, a bromide, and an acetate.

12. The catalyst according to claim 11, wherein said copper containing compound comprises cuprous chloride or cupric chloride.

13. A process for preparation of chloroform and chlorinated paraffins, comprising hydrogenating carbon tetrachloride by one or more n-paraffins in a liquid phase at least at approximately 150° C. in the presence of a catalyst, and separating chloroform product from chlorinated paraffin product, wherein said carbon tetrachloride and said paraffin are provided for hydrogenating at a molar ratio of approximately at least 1:1; wherein said catalyst is present during said hydrogenating in an amount relative to said carbon tetrachloride and said one or more paraffins equal to approximately 1-10% by volume; and wherein said catalyst comprises:
approximately 1.5-4.0% by weight of a copper compound selected from the group consisting of a copper(I) compound and a copper(II) compound;
approximately 30.0-50.0% by weight of a nitrogen-containing organic compound selected from the group consisting of a tertiary ammonium salt, an amino acid, an amide, an alkanolamine, urea, and derivatives thereof; and
a liquid phase base selected from the group consisting of an alcohol, a hydroxyl-containing organic compound, and water.

14. The process according to claim 13, wherein said one or more n-paraffins comprises a mixture of paraffins selected from alkanes having between about 10-20 carbon atoms.

15. The process according to claim 13, wherein said hydrogenating is carried out at said temperature for approximately 3-12 hours.

16. The process according to claim 15, wherein said hydrogenating is carried out at said temperature for approximately 6-8 hours.

17. The process according to claim 16, wherein said temperature is approximately 150-170° C.

18. The process according to claim 17, wherein said carbon tetrachloride and said paraffin are provided for hydrogenating at a molar ratio of approximately between 1:1 to 2:1.

18. The process according to claim 16, wherein said carbon tetrachloride and said paraffin are provided for hydrogenating at a molar ratio of approximately between 1:1 to 2:1.

19. The process according to claim 13, wherein said carbon tetrachloride and said paraffin are provided for hydrogenating at a molar ratio of approximately between 1:1 to 2:1.

20. The process according to claim 13, wherein said nitrogen-containing organic compound is a quaternary ammonium salt of the formula [NR¹R²R³R⁴]⁺X⁻, wherein said R¹, R², R³ and R⁴ are functional groups selected from the group consisting of alkyls, arylalkyls, cycloalkyls, and hydroxyalkyls.

21. The process according to claim 20, wherein said X⁻ is an ion selected from the group consisting of chloride, bromide, and alcoholate.

22. The process according to claim 13, wherein said nitrogen-containing organic compound is an amino acid selected from the group consisting of threonine, asparagine, hydroxyproline, betaine, cysteine, and serine.

23. The process according to claim 13, wherein said nitrogen-containing organic compound is an amide selected from the group consisting of formamide, acetatamide, dimethyl formamide, dimethylacetatcmide, caprolactam.

24. The process according to claim 13, wherein said nitrogen-containing organic compound is urea.

25. The process according to claim 13, wherein said nitrogen-containing organic compound is an alkanolamine selected from the group consisting of ethanolamine, di-ethanolamine, tri-ethanolamine, hydroxides of ethanolamines, and di-(2-hydroxyethyl)dodecylamine.

26. The process according to claim 13, wherein said catalyst comprises 30.0-50.0% by weight of a mixture of at least two nitrogen-containing organic compounds.

27. The process according to claim 13, wherein said liquid phase base is an alcohol selected from the group consisting of methanol, ethanol, and isopropanol.

28. The process according to claim 13, wherein said liquid phase base is an aromatic alcohol selected from the group consisting of phenol and alkylphenol.

29. The process according to claim 13, further comprising separating said chlorinated n-paraffin product according to size of paraffin.

30. The process according to claim 13, wherein said temperature is maintained at approximately 150-170° C.
